(19) United States
(12) Patent Application Publication
(10) Pub. No.: US 2014/0109900 A1
(43) Pub. Date: Apr. 24, 2014

(54) MEDICINAL INHALATION DEVICES, VALVES AND COMPONENTS THEREOF

(71) Applicant: Philip A. Jinks, Loughborough (GB)

(72) Inventor: Philip A. Jinks, Loughborough (GB)

(73) Assignee: 3M INNOVATIVE PROPERTIES COMPANY, St. Paul, MN (US)

(21) Appl. No.: 14/122,704

(22) PCT Filed: Jun. 12, 2012
(86) PCT No.: PCT/US12/42023

§ 371 Date: Nov. 27, 2013

(30) Foreign Application Priority Data
Jun. 15, 2011 (GB) 1110058.3

(51) Int. Cl. A61M 15/00; A61M 16/20

(52) U.S. Cl. CPC A61M 15/009; A61M 15/0065; A61M 16/20
USPC 128/200.23; 29/890.124; 427/2.3

(57) ABSTRACT

A method of making a metered dose valve or a component including the step of forming a coating on at least a portion of a surface of the valve or the component, wherein said coating comprises a particulate material selected from the group consisting of Magnesium Stearate, Calcium Stearate, Zinc Stearate, Aluminium Stearate, Stearic acid, Magnesium Palmitate, Calcium Palmitate, Zinc Palmitate or Aluminium Palmitate, Palmitic acid, and mixtures thereof.

### FIELD OF THE INVENTION

The present invention relates to medicinal inhalation devices, metered dose valves and valve components for such devices as well as methods of making such metered dose valves and components thereof.

### BACKGROUND OF THE INVENTION

Medicinal inhalation devices, in particular pressurized inhalers, such as pressurized metered dose inhalers (pMDIs), are widely used for delivering medicaments.

Pressurized metered dose medicinal inhalation devices typically comprise a plurality of hardware components. In the case of pMDIs, these include metered dose valves including their individual components, such as ferrules, valve bodies, valve stems, tanks, springs retaining cups and seals. Metered dose valves and their components have a number of surfaces, some of which contact or may come into contact with a surface of another component.

Often a needed and/or desired material for a particular component is found to be unsuitable in regard to its surface properties, e.g. surface energy. The use of materials having relatively high surface energy for certain components, e.g. metered dose valves and/or individual components thereof, may have undesirable effects for the operation of movable components of the valve and hence operation of the medicinal inhalation device. For example, the seals in metered dose valves generally have high surface energies due to their rubbery natural. Another example is the relatively high surface energy of acetal polymer for valve stems. Such high surface energies of seals and/or valve stems can bring about a high friction between the valve stem and the seal(s) as they pass along one another during actuation. Such high friction typically impacts the force to fire and the force of return of the valve, which in turn generally has a consequent impact on the uniformity of valve actuation, which then in turn may in some cases impact the uniformity of the medicinal delivery. Other examples of potentially undesirable effects as a result of high friction of surfaces of components passing one another may include undesirable wear of the surfaces and/or an increase in the friction over the lifetime of the device, which may lead to, in a worse case, sticking or even seizing of the valve.

In the past it was common to add surfactants soluble in the liquefied propellant to the aerosol formulation. However the customary surfactants used in CFC-containing PMDIs, i.e. oleic acid, sorbitan trioleate, and lecithin are only partially soluble in the hydrofluoroalkanes, 1,1,1,2-tetrafluoroethane (HFA 134a) and 1,1,1,2,3,3,3-heptafluoropropane (HFA 227), used to replace CFC as propellants in metered dose aerosols. Various other types of excipients have been proposed for use with HFA 134a and/or HFA 227 containing PMDIs, see e.g. EP 0536235, EP 0504 112, EP0605578, U.S. Pat. No. 5,415,853, WO94/21228-9, U.S. Pat. No. 5,492,688, EP 0633019, U.S. Pat. No. 5,502,076, U.S. Pat. No. 5,508,023, and US 2004/101483. However most are unsuccessful and/or undesirable. In regard to the latter, the use of formulation excipients have become generally undesirable. This is related to the fact that regulatory agencies have become more stringent e.g. in regard to toxicology testing and the extent of such testing of such excipients.

Various coatings have been proposed for particular components or surfaces of metered dose inhalers, see e.g. EP 642 992, WO 96/32099, WO 96/32150-1, WO 96/32345, WO 99/42154, WO 02/47829, WO03/024623, WO 02/30498, WO 01/64273, WO 91/64274-5, WO 01/64524, and WO 03/006181. However quite a number are related to coating the aerosol cans, and some valve components that are critical in terms of friction, e.g. seals, normally can not be coated by such methods or coatings.

In some cases, valve components or valve sub-assemblies (for example a valve “core” made up of the valve stem, seal(s), and spring) are siliconized (e.g. tumbling in silicone oil). While this may lower friction between e.g. the valve stem and the seal(s), it has been observed that siliconization may also increase the propensity of medicament deposition on the respective parts.

### SUMMARY OF THE INVENTION

Although a number of approaches have been proposed, there is an ongoing need for metered dose valves and/or components thereof for medicinal inhalation devices (in particular pMDIs) having desirable low friction as well as convenient methods of providing such metered dose valves and components.

In one aspect of the present invention there is provided a method of making a metered dose valve for use in a medicinal inhalation device or a component of a metered dose valve for in use a medicinal inhalation device, wherein at least a portion of a surface of the valve or component, respectively, is to be coated, the method comprising the steps: a) providing the valve or the component, respectively; and b) forming a coating on said at least a portion of a surface of the valve or the component, respectively, wherein said coating comprises a particulate material selected from the group consisting of Magnesium Stearate, Calcium Stearate, Zinc Stearate, Aluminium Stearate, Stearic acid, Magnesium Palmitate, Calcium Palmitate, Zinc Palmitate or Aluminium Palmitate, Palmitic acid, and mixtures thereof.

In another aspect of the present invention there is provided a method of making a metered dose valve for use in a medicinal inhalation device, wherein at least a portion of a surface of a component of the valve is to be coated, the method comprising the steps: a) providing the component of the valve; b) forming a coating on said at least a portion of a surface of the component, wherein said coating comprises a particulate material selected from the group consisting of Magnesium Stearate, Calcium Stearate, Zinc Stearate, Aluminium Stearate, Stearic acid, Magnesium Palmitate, Calcium Palmitate, Zinc Palmitate or Aluminium Palmitate, Palmitic acid, and mixtures thereof; and c) assembling the valve using said coated component and, as applicable, other valve-components.

Favorably, singular parts (e.g. seals or valve stems) or subassemblies are coated. Advantageously, the component is a component that comes into contact with a movable component or is movable during storage or delivery from the medicinal inhalation device. The coating of seals or components comprising a seal (e.g. subassemblies comprising seals) or components coming into contact with a seal have been found particularly advantageous. When assembling a valve, one component part (e.g. just the seal) may have been treated or a number of component parts may have be treated (e.g. just the seal and valve stem) or all the component parts may have been treated.

Surprisingly the coating with the aforesaid particulate material, prior to attachment of the valve to the medicinal container (e.g. aerosol can) of the metered dose inhalation device, provides a favorably durable, low friction surface for desirable valve function.

Without wishing to be bound by theory, it appears for example that during dry coating of seals that small particulates of the selected coating material (e.g. commercial Magnesium Stearate), smear out across elastomeric surfaces and become in part embedded in the surface so that an adherent, low friction coating is presented that is not easily washed off (nor dissolved off due to low solubility) in HFA 134a and/or HFA 227 propellant systems. Accordingly the step of forming the coating on said at least a portion of a surface of the valve or the component, respectively, may desirably comprise contacting said at least a portion of a surface of the valve or the component, as applicable, with said particulate material in dry form to provide said coating. For example, the mixing, tumbling or shaking of seals in dry particulate material has been found to provide generally uniform coating on the exposed surfaces, advantageously both on the bores as well as the faces. Dry coating is a convenient and desirable method of coating seals.

Alternatively the step of forming may desirably comprise contacting said at least a portion of a surface of the valve or the component, as applicable, with particulate material suspended in a fluid and/or material solubilized in a fluid and then removing the fluid to provide particulate material coating (e.g. dipping or spray coating). It has been found that evaporation of a suspension of particulate material (such as sub-micron Magnesium Stearate) from components generally provides an even distribution of powder coated onto the surface facilitated by surface tension in the transient meniscus provided by the selected suspending fluid (e.g. ethanol) which pulls the particles towards the surface of the elastomeric or other material onto which it is coated. Wet coating is a convenient and desirable method of coatings valve stems, springs and subassemblies, such as cores, i.e. subassemblies of the valve stem and seal(s) and, if applicable a spring.

For the provision of favorable lubricating properties, desirably the particulates of the particulate material are flat or plate-like. The particulates of the particulate material desirably have a mass mean aspect ratio (ratio of a particulate's longest dimension to its shortest dimension) equal to or greater than 5, more desirably equal to or greater than 10. Aspect ratio may be determined for example by techniques known in the art such as microscopy with image analysis, or combined use of aerodynamic particle sizing or surface area measurement with microscopy or laser diffraction.

Also in regard to facilitating lubrication and desirable coat formation, it has been found desirable that the particulates of the particulate materials are substantially completely de-agglomerated and/or have low average particle size and/or have higher surface area. Moreover, favorably the particulate material is de-agglomerated such that at least 90% by weight of the particulate material passes or would pass, as applicable, a 325 mesh, more favorably at least 90% by weight of the particulate material passes or would pass, as applicable, a 400 mesh, most favorably at least 95% by weight of that particulate material passes or would pass, a applicable, a 400 mesh. The mass median diameter of the particulates of the particulate material, e.g. as determined by Malvern Laser Diffraction, are favorably generally at most 30 microns, more favorably at most 25 microns, even more favorably at most 20 microns, and most favorably at most 15 microns. Desirably the specific surface area (e.g. a BET specific surface area) of the particulate material is equal to or greater than 2 m²/g, in particular equal to or greater than 3 m²/g.

The present invention also includes the following two aspects:

A metered dose valve of a medicinal inhalation device, wherein at least a portion of a surface of a component of the valve is coated prior to attachment of the valve to a medicinal container of the medicinal inhalation device and wherein said coating comprises a particulate material selected from the group consisting of Magnesium Stearate, Calcium Stearate, Zinc Stearate, Aluminium Stearate, Stearic acid, Magnesium Palmitate, Calcium Palmitate, Zinc Palmitate or Aluminium Palmitate, Palmitic acid, and mixtures thereof.

A medicinal inhalation device comprising a metered dose valve, wherein at least a portion of a surface of a component of the valve is coated, said coating comprising a particulate material selected from the group consisting of Magnesium Stearate, Calcium Stearate, Zinc Stearate, Aluminium Stearate, Stearic acid, Magnesium Palmitate, Calcium Palmitate, Zinc Palmitate or Aluminium Palmitate, Palmitic acid, and mixtures thereof, and wherein the medicinal aerosol formulation filled into the device is essentially free of Magnesium Stearate, Calcium Stearate, Zinc Stearate, Aluminium Stearate, Stearic acid, Magnesium Palmitate, Calcium Palmitate, Zinc Palmitate or Aluminium Palmitate, and Palmitic acid.

Metered dose inhalation devices described herein, in particular pMDIs, are advantageous in that favorable valve performance may be achieved, from the first shot, without having Magnesium Stearate, Calcium Stearate, Zinc Stearate, Aluminium Stearate, Stearic acid, Magnesium Palmitate, Calcium Palmitate, Zinc Palmitate or Aluminium Palmitate, Palmitic acid, and mixtures thereof as a formulation excipient (i.e. essentially free of said materials). This is desirable for the patient in that administration of non-medicinal materials may be minimized and for the pharmaceutical company in that design and formulation work may be simplified.

The above summary of the present invention is not intended to describe each disclosed embodiment or every implementation of the present invention. The description that follows more particularly exemplifies illustrative embodiments. Also further embodiments are described in dependent claims. In several places throughout the application, guidance is provided through lists of examples, which examples can be used individually and in various combinations. In each instance, the recited list serves only as a representative group and should not be interpreted as an exclusive list

### BRIEF DESCRIPTION OF DRAWINGS

The invention will now be described with reference to the accompanying drawings in which:

FIG. 1a represents a schematic cross-sectional view of a pressurized metered dose inhaler known in the art and FIG. 1b represents an enlarged view of a portion of the inhaler.

FIGS. 2 to 5 represent schematic cross-sectional views of further metered dose valves known in the art for use in pressurized metered dose inhalers.

FIGS. 6a to 6c represent force to fire, return force and friction results of a through life testing of pMDIs fitted with valves with components untreated, siliconized and wet coated.

FIG. 7 is a photograph of untreated and a series of dry coated metered dose valve seals.

FIG. 8 is a photograph of an apparatus used to measure Angle of Slip.

FIG. 9 is a box plot of results of Angle of Slip measurements of untreated and a number of dry coated metered dose valve seals.

### DETAILED DESCRIPTION

It is to be understood that the present invention covers all combinations of particular, suitable, desirable, favorable, advantageous and preferred aspects of the invention described herein.

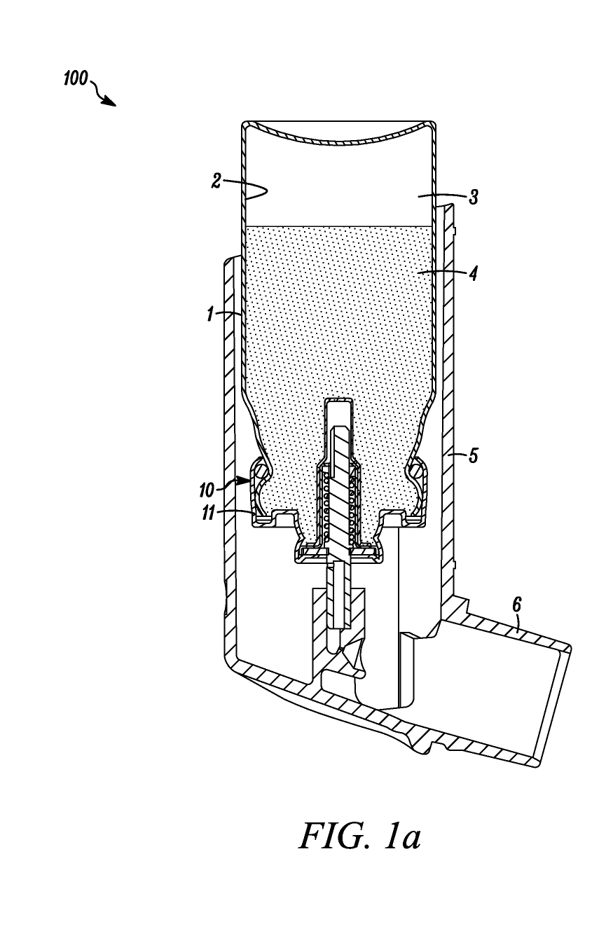

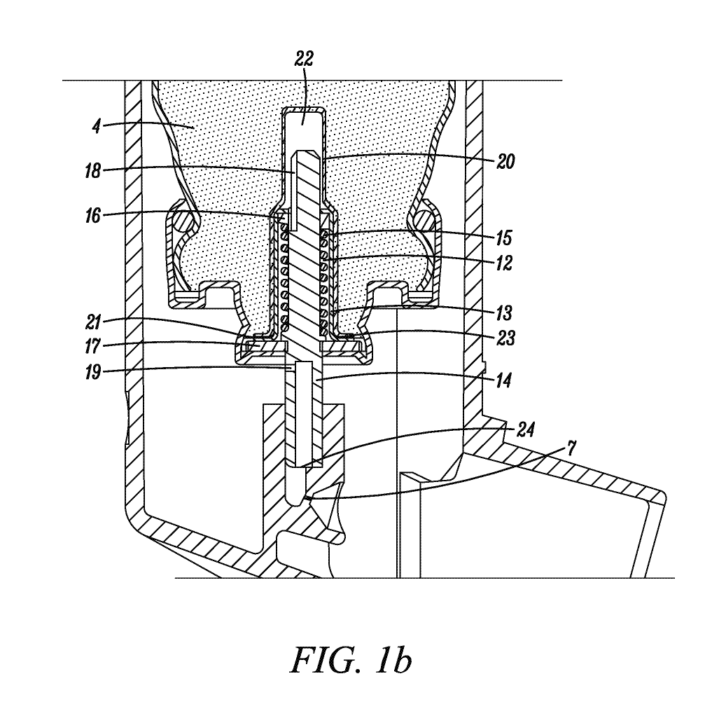

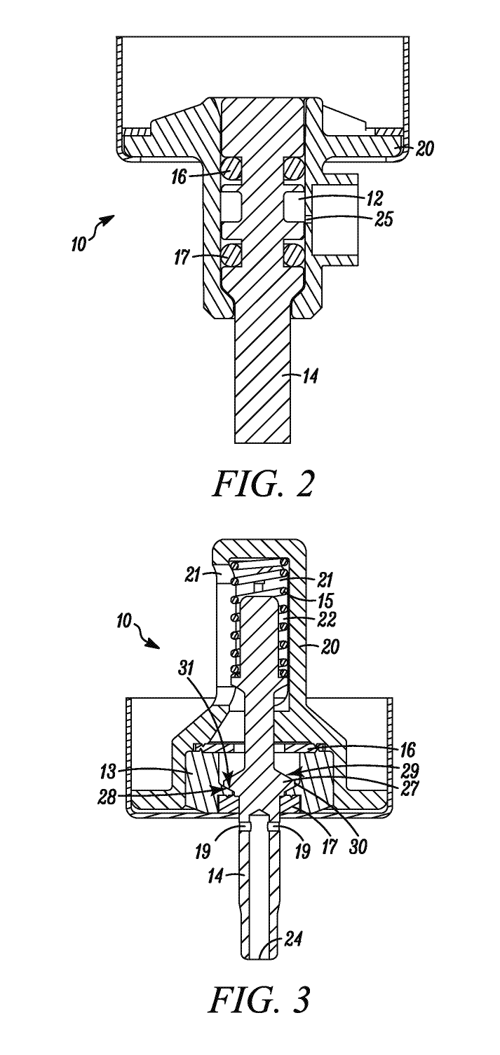

For better understanding of the present invention, in the following an exemplary, well known pressurized metered dose inhaler (FIG. 1) as well as several known metered dose valves for pressurized metered dose inhalers (FIGS. 2 to 5) will be first described. In particular, FIG. 1a shows a metered dose dispenser (100), in particular an inhaler, including an aerosol container (1) fitted with a metered dose valve (10) (shown in its resting position).

Aerosol containers for metered dose inhalers are typically made of aluminum or an aluminum alloy. Aerosol containers may be made of other materials, such as stainless steel, glass, plastic or ceramics.

Returning to FIG. 1a, the valve is typically affixed onto the container via a cap or ferrule (11) (typically made of aluminum or an aluminum alloy) which is generally provided as part of the valve assembly. The illustrated valve is a commercial valve marketed under the trade designation SPRAYMISER by 3M Company, St. Paul, Minn., USA. As shown in FIG. 1a, the container/valve dispenser is typically provided with an actuator (5) including an appropriate patient port (6), such as a mouthpiece. For administration to the nasal cavities the patient port is generally provided in an appropriate form (e.g. smaller diameter tube, often sloping upwardly) for delivery through the nose. Actuators are generally made of a plastic, for example polypropylene or polyethylene. As can be seen from FIG. 1a, the inner walls (2) of the container and the outer walls of the portion(s) of the metered dose valve located within the container defined a formulation chamber (3) in which aerosol formulation (4) is contained. Depending on the particular metered dose valve and/or filling system, aerosol formulation may be filled into the container either by cold-filling (in which chilled formulation is filled into the container and subsequently the metered dose valve is fitted onto the container) or by pressure filling (in which the metered dose valve is fitted onto the container and then formulation is pressure filled through the valve into the container).

An aerosol formulation used in a metered dose inhaler typically comprises a medicament or a combination of medicaments and liquefied propellant selected from the group consisting of HFA 134a, HFA 227 and mixtures thereof.

Medicament may be provided in particulate form (generally having a mass median size in the range of 1 to 10 microns) suspended in the liquefied propellant. Alternatively medicament may be in solution (e.g. dissolved) in the formulation. In the event a combination of two or more medicaments is included, all the medicaments may be suspended or in solution or alternatively one or more medicaments may be suspended, while one or more medicaments may be in solution. A medicament may be a drug, vaccine, DNA fragment, hormone or other treatment. The amount of medicament would be determined by the required dose per puff and available valve sizes, which are typically 25, 50 or 63 microlitres, but may include 100 microlitres where particularly large doses are required. Suitable drugs include those for the treatment of respiratory disorders, e.g., bronchodilators, anti-inflammatories (e.g. corticosteroids), anti-allergics, anti-asthmatics, anti-histamines, and anti-cholinergic agents. Therapeutic proteins and peptides and monoclonal antibodies may also be employed for delivery by inhalation. Exemplary drugs which may be employed for delivery by inhalation include but are not limited to: albuterol, terbutaline, ipratropium, oxitropium, tiotropium, aclidinium, glycopyrronium, beclomethasone, flunisolide, budesonide, mometasone, ciclesonide, cromolyn sodium, nedocromil sodium, ketotifen, azelastine, ergotamine, cyclosporine, salmeterol, fluticasone, formoterol, procaterol, indacaterol, TA2005, vilanterol, omalizumab, zileuton, insulin, pentamidine, calcitonin, leuprolide, alpha-1-antitrypsin, interferons, triamcinolone, and pharmaceutically acceptable salts and esters thereof such as albuterol sulfate, formoterol fumarate, salmeterol xinafoate, vilanterol terfenetate, beclomethasone dipropionate, triamcinolone acetonide, fluticasone propionate, fluticasone furoate, tiotropium bromide, aclidininium bromide, glycopyrronium bromide, leuprolide acetate and mometasone furoate.

Embodiments in accordance with certain aspects of the present invention include metered dose valves or medicinal inhalation devices with metered dose valves, comprising a coating on at least a portion of a surface of a component of the valve. The coating comprises a particulate material selected from the group consisting of Magnesium Stearate, Calcium Stearate, Zinc Stearate, Aluminium Stearate, Stearic acid, Magnesium Palmitate, Calcium Palmitate, Zinc Palmitate or Aluminium Palmitate, Palmitic acid, and mixtures thereof. Advantageously, the coating is on the component(s) prior to fitting the valve to a medicinal container (e.g. aerosol can) of the medicinal inhalation device.

As mentioned above, medicinal inhalation devices described herein, in particular pMDIs, are advantageous in that favorable valve performance may be achieved, from the first shot, whereby the formulation originally filled in to the device is essentially free (less than 0.0001 wt % with respect to the formulation) or more particularly free, of Magnesium Stearate, Calcium Stearate, Zinc Stearate, Aluminium Stearate, Stearic acid, Magnesium Palmitate, Calcium Palmitate, Zinc Palmitate or Aluminium Palmitate, and Palmitic acid.

Embodiments in accordance with the present invention are particularly useful in that the medicinal aerosol formulation may be substantially free of solubilized surfactant (0.005 wt % with respect to the formulation); or is essentially free (less than 0.0001 wt % with respect to the formulation) or free of a solubilized surfactant. Alternatively or additionally, embodiments described in detail below, are particularly useful in metered dose inhalers including a medicinal aerosol formulation that contains low amounts of ethanol (less than 5 wt % with respect to the formulation), or is substantially free (less than 0.1 wt % with respect to the formulation) or free of ethanol. Alternatively, embodiments may include a medicinal aerosol formulation that contains relatively high amounts of ethanol e.g. 5 to 15 wt %.

Embodiments in accordance with the invention may, as desired or needed, comprise other formulation excipients, such as glycerol, ascorbic acid, mineral acid such as hydrochloric acid, CO₂, N₂O or a particulate bulking agent.

The valve shown in FIG. 1a, better viewed in FIG. 1b, includes a metering chamber (12), defined in part by an inner valve body (13), through which a valve stem (14) passes. The valve stem, which is biased outwardly by a compression spring (15), is in sliding sealing engagement with an inner tank seal (16) and an outer diaphragm seal (17). The valve also includes a second valve body (20) in the form of a bottle emptier.

(For the sake of clarity in the description of various metered dose valves, in particular those including at least two valve bodies, in the following a valve body defining in part the metering chamber will be referred to as a “primary” valve body, while other types of valve body, e.g. defining a pre-metering region, a pre-metering chamber, a spring cage and/or a bottle emptier will be referred to as a “secondary” valve body.)

Returning to FIG. 1a, aerosol formulation (4) can pass from the formulation chamber into a pre-metering chamber (22) provided between the secondary valve body (20) and the primary valve body (13) through an annular space (21) between the flange (23) of the secondary valve body and the primary valve body. To actuate (fire) the valve, the valve stem (14) is pushed inwardly relative to the container from its resting position shown in FIGS. 1a and b, allowing formulation to pass from the metering chamber through a side hole (19) in the valve stem and through a stem outlet (24) to an actuator nozzle (7) then out to the patient. When the valve stem (14) is released, formulation enters into the valve, in particular into the pre-metering chamber (22), through the annular space (21) and thence from the pre-metering chamber through a groove (18) in the valve stem past the tank seal (16) into the metering chamber (12).

As mentioned above, FIGS. 2 to 5 show other known metered dose valves used in pMDIs. Similar to the valve shown in FIG. 1, the valves of FIGS. 2 to 5 are typically fitted via a ferrule onto an aerosol container whereby a formulation chamber is defined by the inner walls of the container and the outer walls of the portion(s) of the valve located within the container. For the sake of ease in understanding and comparison, similar components of the respective valves are identified with like reference numbers in the Figures.

FIG. 2 shows a metered dose valve (10) of a type generally similar to that disclosed and described in U.S. Pat. No. 5,772,085 (incorporated herein by reference). The valve is shown in its resting position and includes a valve body (20) and a valve stem (14). The valve stem, which is biased outwardly under the pressure of the aerosol formulation contained within the formulation container, is provided with an inner seal and an outer seal (16 and 17). Unlike the valves in FIG. 1 and FIGS. 3 to 5, which are push-to-fire type valves, the valve here is a release-to-fire type valve. To actuate the valve, the valve stem (14) is first pushed upwards into the formulation chamber (not shown), so that the outer seal (17) passes inwardly beyond an outlet (25) provided in the external portion of the valve body and the inner seal (16) then passes inwardly and disengages from the inner walls of the valve body, thus bringing the metering chamber (12) up into the formulation chamber so that formulation can enter the metering chamber (referred to as the priming position of the valve) and then the valve stem is released moving outwardly so that the inner seal re-engages the valve body and the outer seal then passes outwardly beyond the outlet, bringing the metering chamber in communication with the outlet, so that formulation passes through the outlet to the patient.

FIG. 3 shows a metered dose valve (10) of the type generally similar to that disclosed and described in WO 2004/022142 (incorporated herein by reference). The valve is shown in its resting position and includes a secondary valve body (20) and a valve stem (14) that is biased outwardly by a compression spring (15). The valve is provided with an inner seal (16) and outer diaphragm seal (17), with the valve stem being in sliding sealing engagement with the diaphragm seal. In this valve, the secondary valve body is in the form of a spring cage housing having three slots (21, two visible) providing communication between the formulation chamber (not shown) and a pre-metering chamber (22). This valve includes a transitory metering chamber formed upon actuation of the valve. During actuation of the valve, as the valve stem (14) is pushed inwardly relative to the container, a metering chamber (12, not visible) is formed between a lower surface (28) of a conical portion (27) of the valve stem (14) and an upper, sloping surface (31) of a primary valve body (13). Aerosol formulation passes around the shoulder (30) of the conical portion of the valve stem into the forming metering chamber and as the valve stem is further pushed in the upper surface (29) of the conical portion forms a face seal with the inner seal (16), thereby sealing off the metering chamber. As the valve stem is yet further displaced inwardly, formulation is allowed to pass from the metering chamber through side holes (19) in the valve stem and through a stem outlet (24) in the valve stem, and subsequently out to the patient typically via an actuator nozzle (7, not shown).

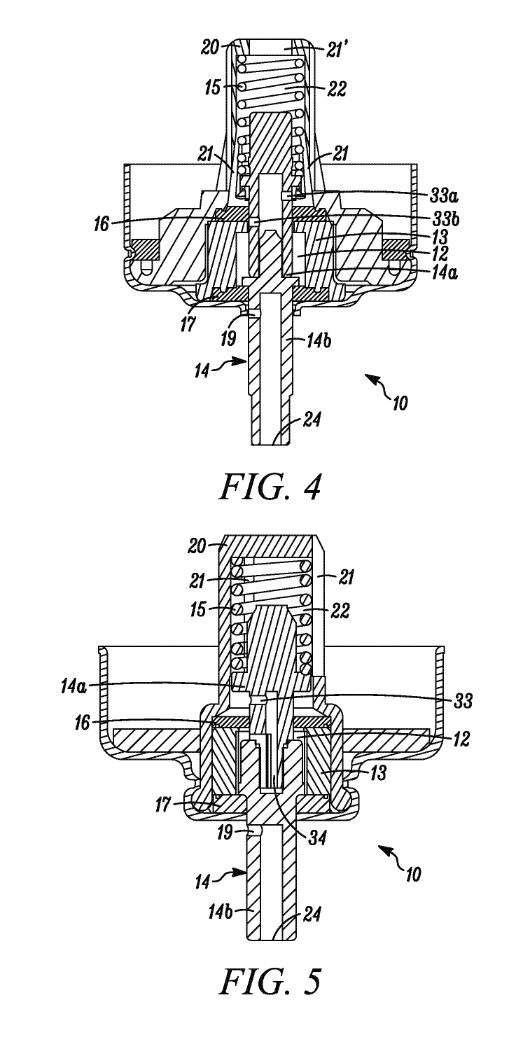

FIG. 4 shows a commercial metered dose valve supplied by Bespak, Bergen Way, King's Lynn, Norfolk, PE30 2JJ, UK under the trade designation BK357, in its resting position. The valve includes a secondary valve body (20) in the form of a spring cage with two slots (21) and an opening at the top (21′) allowing communication between the formulation chamber (not shown) and a pre-metering chamber (22). The valve also includes a valve stem (14), made of two components (14a, 14b), which is biased outwardly by a compression spring (15) and passes through a metering chamber (12) defined in part by a primary valve body (13). The valve stem is in sliding sealing engagement with an inner seal (16) and an outer diaphragm seal (17). Aerosol formulation can pass from the pre-metering chamber (22) into the metering chamber (12) via side holes (33a, 33b) in the upper portion (14a) of the stem (14). Similar to the valve shown in FIG. 1, to actuate (fire) the valve, the valve stem (14) is pushed inwardly relative to the container, allowing a metered dose of formulation to pass from the metering chamber through a side hole (19) in the valve stem and through a stem outlet (24) and then typically through an actuator nozzle (7, not shown) out to the patient.

FIG. 5 shows a commercial metered dose valve supplied by Valois SAS, Pharmaceutical Division, Route des Falaises, 27100 le Vaudreuil, France under the trade designation RCS, in its resting position. The valve includes a secondary valve body (20) in the form of a spring cage with three slots (21, two visible) allowing communication between the formulation chamber (not shown) and a pre-metering chamber (22). The valve also include a valve stem (14), made of two components (14a, 14b), which is biased outwardly by a compression spring (15) and passes through a metering chamber (12) defined in part by a primary valve body (13). The valve stem is in sliding sealing engagement with an inner seal (16) and an outer diaphragm seal (17). Aerosol formulation can pass from the pre-metering chamber (22) into the metering chamber through a side hole (33) and an internal channel (34) provided in the upper portion (14a) of the valve stem. Similar to the valve shown in FIG. 1, to actuate (fire) the valve, the valve stem (14) is pushed inwardly relative to the container, allowing formulation to pass from the metering chamber through a side hole (19) in the valve stem and through a stem outlet (24) and then typically through an actuator nozzle (7, not shown) out to the patient.

With the exception of the elastomeric seals used in metered dose valves, typically the components of such valves are made of metal (e.g. stainless steel, aluminum or aluminum alloy) or plastic. For example compression springs are generally made of a metal, in particular stainless steel as the conventional material. Compression springs may also be made of aluminum or aluminum alloy. Valve stems and valve bodies are generally made of metal and/or plastic; as a metal conventionally stainless steel is used (other metals that may be used include aluminum, aluminum alloy and titanium) and as plastics conventionally polybutylene terephthalate (PBT) and/or acetal are used (other polymers that may be used include polyetheretherketones (PEEK), polymethylpentene, polyphenylene sulphide, thermotropic liquid crystalline polymer, PTFE or nylon, other polyesters (such as tetrabutylene terephthalate), polycarbonates and polyethylene).

Seals are typically elastomeric. Seals are generally made of polybutadiene-acrylonitrile (Nitrile) polymer, polychloroprene (Neoprene), polyethylene-propylene-diene-modified (EPDM), polyisobutylene-isoprene (Butyl), or chlorinated polyisobutylene-isoprene (Chlorobutyl), each compounded with suitable fillers cross-linking agents and processing aids. Seals may also be compounded from thermoplastic elastomeric materials, e.g. Flexomer® DFDB1085(ex Dow), Santoprene® (ex Advanced Elastomer Systems), or mixtures of thermosetting elastomers with thermoplastic materials.

Embodiments in accordance with other aspects of the present invention include forming a coating on at least a portion of a surface of a metered dose valve or a component thereof, where the coating comprises a particulate material selected from the group consisting of Magnesium Stearate, Calcium Stearate, Zinc Stearate, Aluminium Stearate, Stearic acid, Magnesium Palmitate, Calcium Palmitate, Zinc Palmitate or Aluminium Palmitate, Palmitic acid, and mixtures thereof. Suitable stearates, stearic acids, palmitates, palmitic acids for use are commercially available. Typically commercial stearates include in part palmitates.

Desirably a component treated in accordance with aspects of the present invention described herein is a component of a metered dose valve used in a metered dose inhaler, in particular in a pMDI. Favorably at least a portion of a surface, more favorably the entire surface, of a component or components of a metered dose valve, which either come into contact with a movable component or are movable during storage or delivery from the medicinal inhalation device are treated according to methods described herein. Examples of such components for metered dose valves include e.g. seals, valve bodies, valve stems or compression springs of metered dose valves. More favorably said component may be selected from the group consisting of a valve body (e.g. a primary and/or a secondary valve body), a compression spring, a valve stem, a seal (e.g. inner and/or outer seal), and sub-assemblies comprising two or more of the aforesaid parts. Most favorably the component is a seal, a component that comes into sliding contact with the seal (e.g. valve stem or valve body, depending on the particular design of the metered dose valve) or a component comprising a seal (e.g. a core made of the valve stem, and one or more seals and if applicable a spring).

One type of singular component part or parts, e.g. seal or seals, may be treated or a mixture of singular component parts (e.g. valve stems and springs) made be treated together. Alternatively or additionally, as applicable, sub-assembly component(s) or in according to one aspect of present invention the whole valve itself may be treated.

In assembling a valve using a treated component to provide an assembled valve for attachment to the medicinal container (e.g. aerosol can of a pMDI) all the component parts making up the valve may be coated or more commonly only a certain part or certain parts of valve may be coated.

The step of forming a coating may comprises contacting said at least a portion of a surface of the valve or the component, as applicable, with particulate material in dry form to provide said coating (dry coating); or contacting said at least a portion of a surface of the valve or the component, as applicable, with particulate material suspended in a fluid and/or material solubilized in a fluid and then removing the fluid to provide particulate material coating (wet coating).

Dry coating may be conducted by mixing (e.g. shaking, tumbling, stirring) component(s) in the dry particulate material. It has been found that as increasing amounts of coating powder are applied, the surfaces facing outwardly, i.e. easy accessible surfaces, become so to say saturated with powder at a particular level of powder, leaving free powder to accumulate on the less accessible surfaces (e.g. the bore of a seal) of the component. Hence, when larger amounts of coating powder are applied, an increasingly larger proportion coats the less accessible surfaces until more or less a plateau of coating is reached. (Any excess particulate material may be removed by e.g. sifting.) Surprisingly it has been found that such less accessible surfaces, e.g. bores of seals, often receive a more thorough and uniform coating (i.e. a higher coating density) than easy accessible surfaces, e.g. faces and outer edges of seals, possibly due to a lack of or a minimal amount of “buffeting” of components against less accessible surfaces of other components during coating and/or, as applicable for example in the case of wet coating, an art turbophoresis effect.

Wet coating may be conducted using a dispersion of the particulate material in the fluid or partially or completely solubilized material in fluid. Fluids used in wet coating may include water, a hydrocarbon, an alcohol, an ester, a fluorocarbon or a mixture thereof, in particular a volatile hydrocarbon, a volatile alcohol, a volatile ester, a volatile fluorocarbon or a mixture thereof, more particularly n-hexane, n-heptane, ethanol, isopropanol, ethylacetate, HFE 7100 or a mixture thereof. Where material is solubilized in a solvent, e.g. hot ethanol, the coating on the surface is provided by drainage/evaporation of the solvent. This approach may provide a particulate material coating having a glassy appearance and/or properties. Generally however it has been found desirable to use dispersions of particulate material for wet coating. In particular it has been found that concentrations of particulate material dispersions or solutions equal to or greater than 0.1% w/v desirably facilitate film formation and films that provide a lubricating effect after removal of the fluid (e.g. drainage/evaporation of the fluid). Dispersions at concentrations greater than 25% w/v are generally impractical, the dispersion being quite viscous.

As indicated supra, for the provision of a coating having desirable lubricating properties, it is advantageous that the particulates of the particulate material are flat or plate-like.

For both wet and dry coating for desirable coat formation and/or subsequent coat properties, it has been found desirable that the particulates of the particulate materials are substantially completely de-agglomerated. For example, for wet coating such de-agglomeration of the suspended particulate material can be advantageously assured prior to contacting the component or components to the coating dispersion of particulate material, for example by high shear mixing the suspending fluid using a Silverson mixer while adding the particulate material. For dry coating and, as applicable, for wet coating, favorably at least 90% by weight of the particulate material passes or would pass, as applicable, a 325 mesh, in particular at least 90% by weight of the particulate material passes or would pass, as applicable, a 400 mesh, more particularly at least 95% by weight of that particulate material passes or would pass, a applicable, a 400 mesh.

Additionally or alternatively, the mass median diameter of the particulates of the particulate material, e.g. as determined by Malvern Laser Diffraction, are favorably generally at most 30 microns, more favorably at most 25 micron, even more favorably even more favorably at most 20 micron and most favorably at most 15 micron. For wet coating, methods may advantageously include a process step or steps, such as high pressure homogenization, to produce a dispersion comprising submicron particulates (i.e. having a mass median diameter less than 1 micron).

Additionally or alternatively the specific surface area, e.g. as determined by a method based on the theory of Brunauer, Emmett and Teller, J. Am. Chem. Soc., vol. 60, p. 577 (1960), e.g. using a Beckman Coulter SA 3100 Surface area and Pore size analyzer “BET specific surface area”, of the particulate material is desirably equal to or greater than 2 m²/g, in particular greater than 3 m²/g.

Magnesium stearate and mixtures of magnesium stearate and magnesium palmitate are particularly preferred. Particulate material comprising crystalline magnesium stearate and/or palmitate are particularly preferred, in particular crystalline magnesium stearate and/or palmitate include an generally long lattice spacing, e.g. a d-spacing equal to or greater than 10, e.g. as determined by X-ray diffraction studies.

### Experimental Section

### Experiment 1—Wet Coating:

10 g of Magnesium Stearate (NF ex FISCHER SCIENTIFIC Lot 432021, ˜10 micron, stearic acid ≧40%; total of stearic and palmitic acid >90%) was added to dehydrated ethanol (400 g) and high shear mixed using a Silverson mixer for 1 minute. The dispersion was added to a product vessel of an Avestin C50 homogenizer and processed at 20,000 p.s.i. using re-circulation for 30 minutes. Microscopic analysis of a sample taken from the resulting dispersion indicated that particulate material has a relatively uniform particle size of about 0.5 μm.

A plurality of valve cores (sub-assemblies consisting of stainless steel valve stem, a stainless steel valve spring, an outer diaphragm nitrile seal and an inner tank nitrile seal (see FIGS. 1a&b) were dipped in the prepared dispersion and then allowed to drain on a tray while letting the solvent evaporate.

Metered dose valves of the type shown in FIGS. 1a & b were assembled from the coated cores and the other necessary, non coated component parts, i.e. metering tank, bottle emptier and, ferrule.

Inhalers were prepared by cold-filling formulation consisting of 1.97 mg/ml Albuterol Sulfate (having a majority of particles in the range of 1 to 3 microns) and HFA 134a into 10 ml aluminum cans, crimping the assembled valves to the cans and finally allowing the inhalers to warm to room temperature. The so-prepared inhalers were tested for force to fire, return force and friction as described below.

As a comparative, metered dose inhalers with siliconized (dimethicone) valves were prepared.

### Method of Force Measurement

After allowing a period of 7 days for acclimatization of the seals in the aerosol units, the following is test method used for the force characteristics of the valve after actuating various numbers of doses through the life of the unit as prescribed below:

1. Insert the aerosol unit into a fresh actuator, and prime the inhaler, i.e. shake the inhaler with a gentle rocking action through 180° inversion for at least 10 seconds and immediately fire two shots to waste.

2. Weigh the aerosol unit with actuator, then fire one shot. Repeat five times, followed by a further weighing, and calculate five shot weights by subtracting weighings.

3. Fire two priming shots. Insert aerosol unit into a tensile tester and record the profile of force against valve travel for firing and return of the valve at 20 mm/min. Record the Force to fire the valve, corresponding to the point in the cycle where the stem side hole first breaks through the elastomeric outer seal seal during its inward travel. Record the Return Force for the valve, corresponding to the point in the cycle where the stem groove first breaks through the tank seal during its outward travel. Friction represents half the difference in outward and return forces at a prescribed valve stem travel.

4. Repeat step 3, two more times.

5. Fire 40 shots to waste using an automatic valve firing machine.

6. Repeat steps 1 to 5.

7. Repeat steps 1 to 4, then fire 40 shots to waste using an automatic valve firing machine.

8. Repeat steps 1 to 4.

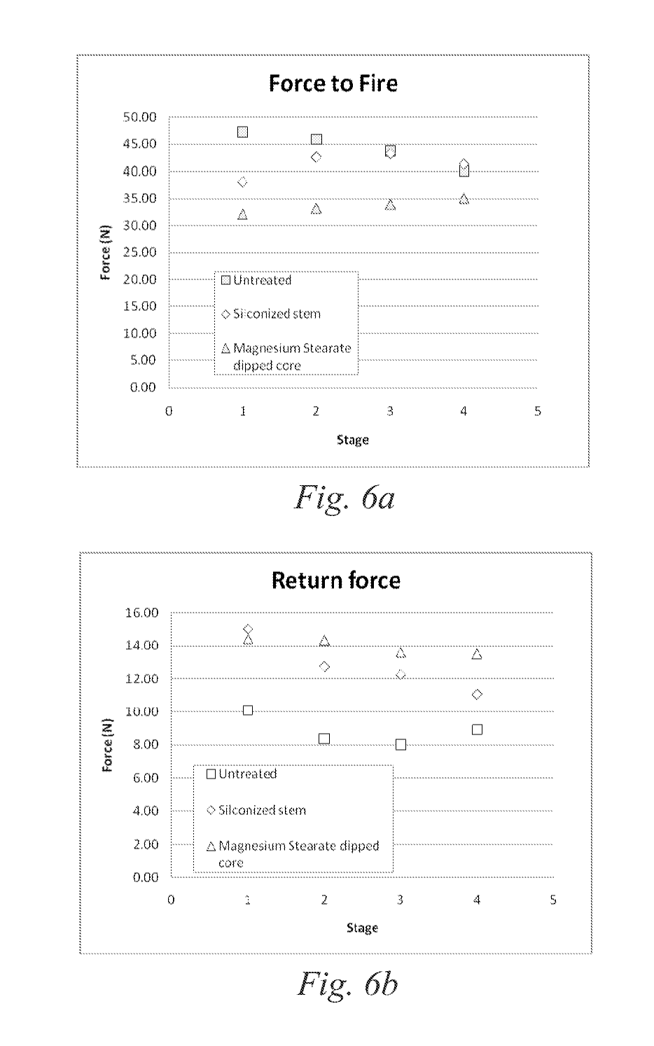

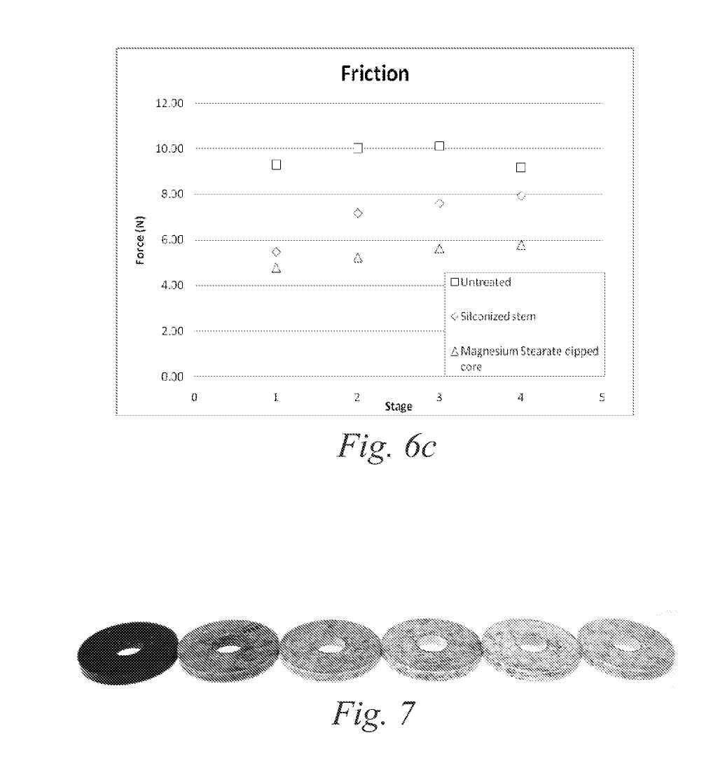

The four occasions that steps 1 to 4 are carried out are referred to as Stages 1 to 4 and represent shot umbers 22-30, 78-86, 134-142 and 190-198 respectively. The results are summarized in FIG. 6 a to c; □ untreated; ⋄ siliconized; and Δ magnesium stearate wet coated.

### Experiment 2—Dry Coating:

Elastomer outer diaphragm nitrile seals (total weight 57 grams) were placed in a 250 ml beaker, and dry particulate Magnesium Stearate ((vegetable grade) PARTEC™ LUB MST ex MERCK Lot K41295363043; mass median diameter of particles about 5 microns.) in an amount as indicated in the following Table was added.

TABLE 1

| | Weight of | Whiteness | Whiteness | Whiteness |
|---|---|---|---|---|
| | powder | of seal | of seal | of seal |
| No. | (mg) | bore | edge | face |
| 2a | 65 | 83 ± 12 | 109 ± 15 | 104 ± 19 |
| 2b | 97 | 143 ± 14 | 158 ± 20 | 147 ± 17 |
| 2c | 130 | 176 ± 11 | 172 ± 21 | 158 ± 24 |
| 2d | 152 | 191 ± 8 | 184 ± 21 | 171 ± 20 |
| 2e | 165 | 184 ± 8 | 174 ± 23 | 156 ± 24 |
| no | — | 28 ± 5 | 30 ± 2 | 19 ± 3 |
| coating | | | | |

The beaker was closed with a film sold under the trademark PARAFILM, and the beaker was shaken for 30 seconds Thereafter, the coated seals were removed from the beaker. Coated seals and uncoated seals were photographed. The appearance of the elastomeric outer seal components is exemplified in the photograph shown in FIG. 7, which shows, from left to right, uncoated, then coated with Magnesium Stearate at levels of 65, 97, 130, 152 and 165 mg respectively.

The level of whiteness given in Table 1 was measured using the following technique: The electronic photographs were opened in Corel Paint Shop Pro Photo X2 software using default settings. Ensure selection of Image>greyscale. Open the ‘tools’ toolbar and select the Dropper tool. In the Dropper palette, select 11×11 pixel size. Sample 10 colour readings from regions where the camera flash was directed in the bore of the coated components. The R, G and B readings will be equal for each reading, due to selection of the greyscale option. The readings represent “whiteness” on a scale of 0 to 255, and provide an indication of the extent of coating. The tabulated values show average whiteness together with the standard deviation of a set of 10 readings, each value representing the averaged results for 5 components.

Additional dry coated seals were prepared using Magnesium Stearate from FISCHER SCIENTIFIC (NF, Lot 432021) using the method described above with the amounts of Magnesium Stearate given in Table 2:

TABLE 2

| No. | Weight of powder (mg) |
|---|---|
| 2f | 65 |
| 2g | 130 |

### Angle of Slip

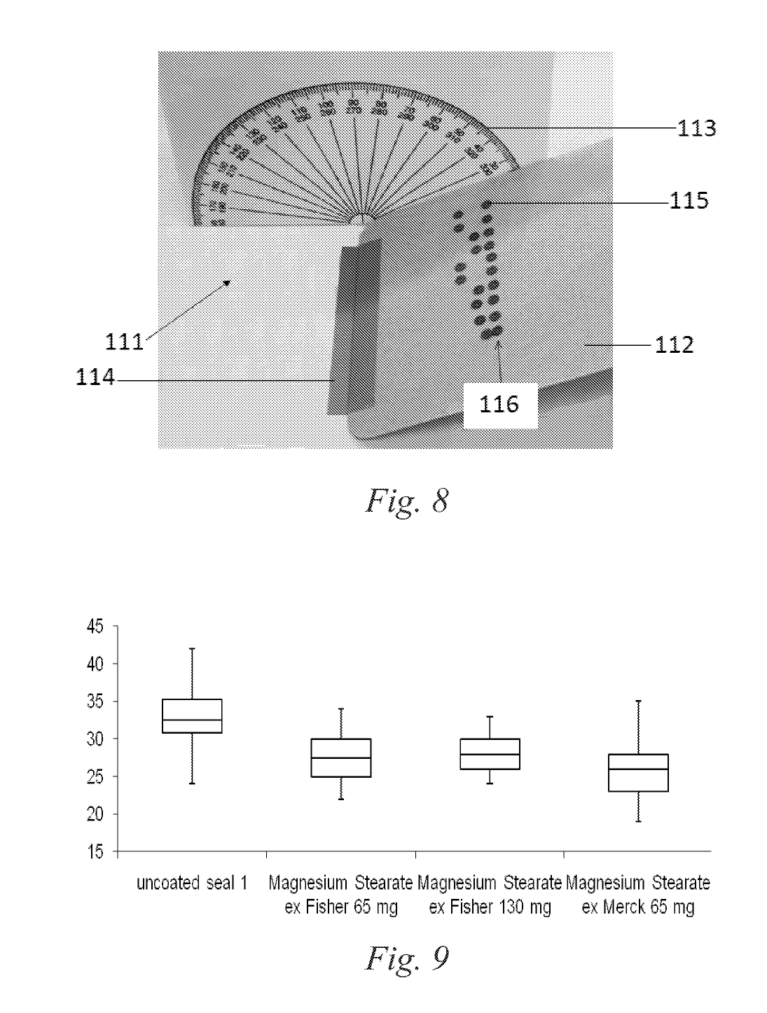

Uncoated seals and seals from Example No. 2a, 2f and 2g were measured for Angle of Slip using the apparatus (111) shown in FIG. 8 and the following method: In the apparatus, a clipboard (112) was adhered at one edge to a flat horizontal surface by means of adhesive tape (114), to provide a hinge, and a protractor (113) is mounted vertically and orthogonally to the hinge line which intersects the central point on the protractor. Ten test seals (115) were affixed using double-sided adhesive tape in a line (116) at some distance from and parallel to the hinge. Thereafter one of the same batch of exemplary seals were mounted on top of each of the ten affixed seals (e.g. a 2a-exemplary seal was placed loose on a fixed 2a-exemplary seal). The clipboard was pivoted about the hinge by 1 degree increments with a 5 seconds dwell time at each new angle increment, and the angle at which each top seal slips off the seal underneath it was recorded. FIG. 9 shows a box-plot of the results.

1. A method of making a metered dose valve for use in a medicinal inhalation device or a component of a metered dose valve for use in a medicinal inhalation device, wherein at least a portion of a surface of the valve or component, respectively, is to be coated, the method comprising the steps:
a) providing the valve or the component, respectively; and
b) forming a coating on said at least a portion of a surface of the valve or the component, respectively, wherein said coating comprises a particulate material selected from the group consisting of Magnesium Stearate, Calcium Stearate, Zinc Stearate, Aluminium Stearate, Stearic acid, Magnesium Palmitate, Calcium Palmitate, Zinc Palmitate or Aluminium Palmitate, Palmitic acid, and mixtures thereof.

2. A method of making a metered dose valve for use in a medicinal inhalation device, wherein at least a portion of a surface of a component of the valve is to be coated, the method comprising the steps:
a) providing the component of the valve;
b) forming a coating on said at least a portion of a surface of the component, wherein said coating comprises a particulate material selected from the group consisting of Magnesium Stearate, Calcium Stearate, Zinc Stearate, Aluminium Stearate, Stearic acid, Magnesium Palmitate, Calcium Palmitate, Zinc Palmitate or Aluminium Palmitate, Palmitic acid, and mixtures thereof; and
c) assembling the valve using said coated component and, as applicable, other valve-components.

3. A method according to claim 1, wherein the component is a singular part or a sub-assembly.

4. A method according to claim 1, wherein the component is a component that comes in contact with a movable component or is movable during storage or delivery from the medicinal inhalation device.

5. A method according to claim 1, wherein the component is a seal or the component comprises a seal or the component is a component which comes into contact with a seal, in particular the component is a seal or a component comprising a seal.

6. A method according to claim 1, wherein the component is a seal and comprises Nitrile, EPDM, Neoprene, Butyl, Chlorobutyl or a thermoplastic eleastomer.

7. A method according to claim 1, wherein the component is a valve stem, in particular a valve stem made of a material comprising Stainless Steel, Polyoxymethylene, Polybutylene terephthalate, Nylon, PEEK, polymethylpentene, polyphenylene sulphide, thermotropic liquid crystalline polymer or PTFE.

8. A method according to claim 1, wherein the component is a spring, in particular a spring made of Stainless steel.

9. A method according to claim 1, wherein the component is a sub-assembly comprising a valve stem and one or more seals, in particular said sub-assembly further comprises a spring.

10. A method according to claim 1, wherein the step of forming a coating on said at least a portion of a surface of the valve or the component, respectively, comprises contacting said at least a portion of a surface of the valve or the component, as applicable, with particulate material in dry form to provide said coating; or
contacting said at least a portion of a surface of the valve or the component, as applicable, with particulate material suspended in a fluid and/or solubilized in a fluid and then removing the fluid to provide said coating.

11. A method according to claim 10, wherein the fluid comprises water, a hydrocarbon, an alcohol, an ester, a fluorocarbon or a mixture thereof, in particular the fluid comprises a volatile hydrocarbon, a volatile alcohol, a volatile ester, a volatile fluorocarbon or a mixture thereof, more particularly the fluid comprises n-hexane, n-heptane, ethanol, isopropanol, ethylacetate, HFE 7100 or a mixture thereof.

12. A method according to claim 1, wherein the particulate material is de-agglomerated, such that at least 90% by weight of the particulate material passes or would pass, as applicable, a 325 mesh, in particular at least 90% by weight of the particulate material passes or would pass, as applicable, a 400 mesh, more particularly at least 95% by weight of that particulate material passes or would pass, as applicable, a 400 mesh; and/or
the mean median diameter of the particulates of the particulate material is at most 30 microns, in particular at most 25 microns, more particularly at most 20 microns, even more particularly at most 15 microns; and/or
the specific surface area of the particulate material is equal to or greater than 2 m²/g, in particular equal to or greater than 3 m²/g; and/or
the particulate material is flat or plate-like, in particular the mass mean aspect ratio of the particulates of the particulate material is equal to or greater than 5, more desirably equal to or greater than 10.

13. A method according to claim 1, wherein the particulate material comprises Magnesium stearate, Magnesium palmitate or mixtures thereof.

14. A method according to claim 13, wherein the particulate material comprises crystalline Magnesium stearate, Magnesium palmitate or mixtures thereof, in particular crystalline Magnesium stearate, Magnesium palmitate or mixtures thereof having a d-spacing equal to or greater than 10.

15. A method according to claim 1, wherein the medicinal inhalation device is a pressurized metered dose medicinal inhalation device, in particular a pressurized metered dose inhaler.

16. A metered dose valve for use in a medicinal inhalation device, wherein at least a portion of a surface of a component of the valve is coated prior to attachment of the valve to an medicinal container of the medicinal inhalation device and wherein said coating comprises a particulate material selected from the group consisting of Magnesium Stearate, Calcium Stearate, Zinc Stearate, Aluminium Stearate, Stearic acid, Magnesium Palmitate, Calcium Palmitate, Zinc Palmitate or Aluminium Palmitate, Palmitic acid, and mixtures thereof.

17. A valve according to claim 16, wherein the component is a singular part or a sub-assembly.

18. A valve according to claim 16, wherein the component is a component that comes in contact with a movable component or is movable during storage or delivery from the medicinal inhalation device.

19. A valve according to claim 16, wherein the component is a seal or comprises a seal or is a component which comes into contact with a seal, in particular the component is a seal or a component comprising a seal.

20. A valve according to claim 16, wherein the component is a seal and comprises Nitrile, EPDM, Neoprene, Butyl, Chlorobutyl or a thermoplastic eleastomer.

21. A valve according to claim 16, wherein the component is a valve stem, in particular a valve stem made of a material comprising Stainless Steel, Polyoxymethylene, Polybutylene terephthalate, Nylon, PEEK, polymethylpentene, polyphenylene sulphide, thermotropic liquid crystalline polymer or PTFE.

22. A valve according to claim 16, wherein the component is a spring, in particular a spring made of Stainless Steel.

23. A valve any to claim 16, wherein the component is a sub-assembly comprising a valve stem and one or more seals, in particular said sub-assembly further comprises a spring.

24. A valve according to claim 16, wherein the particulate material is de-agglomerated, such that at least 90% by weight of the particulate material passes or would pass, as applicable, a 325 mesh, in particular at least 90% by weight of the particulate material passes or would pass, as applicable, a 400 mesh, more particularly at least 95% by weight of that particulate material passes or would pass, a applicable, a 400 mesh; and/or
the mean median diameter of the particulates of the particulate material is at most 30 microns, in particular at most 25 microns, more particularly at most 20 microns, even more particularly at most 15 micron; and/or
the specific surface area of the particulate material is equal to or greater than 2 m²/g, in particular equal to or greater than 3 m²/g;
the particulates of the particulate material are flat or plate-like, in particular the mass mean aspect ratio of the particulates of the particulate material is equal to or greater than 5, more desirably equal to or greater than 10; and/or
wherein the particulate material comprises Magnesium stearate, Magnesium palmitate or mixtures thereof, in particular crystalline Magnesium stearate, Magnesium palmitate or mixtures thereof, more particularly crystalline Magnesium stearate, Magnesium palmitate or mixtures having a d-spacing equal to or greater than 10.

25. A valve according to claim 16, wherein the medicinal inhalation device is a pressurized metered dose medicinal inhalation device, in particular a pressurized metered dose inhaler.

26. A medicinal inhalation device comprising a metered dose valve, wherein at least a portion of a surface of a component of the valve is coated, said coating comprising a particulate material selected from the group consisting of Magnesium Stearate, Calcium Stearate, Zinc Stearate, Aluminium Stearate, Stearic acid, Magnesium Palmitate, Calcium Palmitate, Zinc Palmitate or Aluminium Palmitate, Palmitic acid, and mixtures thereof, and wherein the medicinal aerosol formulation filled into the device is essentially free of Magnesium Stearate, Calcium Stearate, Zinc Stearate, Aluminium Stearate, Stearic acid, Magnesium Palmitate, Calcium Palmitate, Zinc Palmitate or Aluminium Palmitate, and Palmitic acid.

27. A device according to claim 26, wherein the medicinal aerosol formulation is free of Magnesium Stearate, Calcium Stearate, Zinc Stearate, Aluminium Stearate, Stearic acid, Magnesium Palmitate, Calcium Palmitate, Zinc Palmitate or Aluminium Palmitate, and Palmitic acid.

28. A device according to claim 26, wherein the medicinal aerosol formulation comprises a medicament or a combination of medicaments and liquefied propellant selected from the group consisting of HFA 134a, HFA 227 and mixtures thereof.

29. A device according to claim 26, wherein the medicament of the medicinal aerosol formulation comprises albuterol, terbutaline, ipratropium, oxitropium, tiotropium, aclidinium, glycopyrronium, beclomethasone, flunisolide, budesonide, mometasone, ciclesonide, cromolyn sodium, nedocromil sodium, ketotifen, azelastine, ergotamine, cyclosporine, salmeterol, fluticasone, formoterol, procaterol, indacaterol, TA2005, vilanterol, omalizumab, zileuton, insulin, pentamidine, calcitonin, leuprolide, alpha-1-antitrypsin, interferons, triamcinolone, and pharmaceutically acceptable salts and esters thereof.

30. A device according to claim 26, wherein the medicinal aerosol formulation is substantially free of solubilized surfactants, in particular essentially free of solubilized surfactants, more particularly free of solubilized surfactants; and/or
wherein the medicinal aerosol formulation contains less than 5 wt % with respect to the formulation of ethanol, in particular the medicinal aerosol formulation is substantially free of ethanol, more particularly the medicinal aerosol formulation is free of ethanol.

31. A device according to claim 26, wherein the component is a singular part or a sub-assembly.

32. A device according to claim 26, wherein the component is a seal or comprises a seal or is a component which comes into contact with a seal, in particular the component is a seal or a component comprising a seal.

33. A device according to claim 26, wherein the component is a seal and comprises Nitrile, EPDM, Neoprene, Butyl, Chlorobutyl or a thermoplastic eleastomer.

34. A device according to claim 26, wherein the component is a valve stem, in particular a valve stem made of a material comprising Stainless Steel, Polyoxymethylene, Polybutylene terephthalate, Nylon, PEEK, polymethylpentene, polyphenylene sulphide, thermotropic liquid crystalline polymer or PTFE.

35. A device according to claim 26, wherein the component is a spring, in particular a spring made of Stainless Steel.

36. A device according to claim 26, wherein the component is a sub-assembly comprising a valve stem and one or more seals, in particular said sub-assembly further comprises a spring.

37. A device according to claim 26, wherein the particulate material is de-agglomerated, such that at least 90% by weight of the particulate material passes or would pass, as applicable, a 325 mesh, in particular at least 90% by weight of the particulate material passes or would pass, as applicable, a 400 mesh, more particularly at least 95% by weight of that particulate material passes or would pass, a applicable, a 400 mesh; and/or
the mean median diameter of the particulates of the particulate material is at most 30 microns, in particular at most 25 microns, more particularly at most 20 microns, even more particularly at most 15 micron; and/or
the surface area of the particulate material is equal to or greater than 2 m²/g, in particular equal to or greater than 3 m²/g; and/or
the particulates of the particulate material are flat or plate-like, in particular the mass median aspect ratio of particulates of the particulate material is equal to or greater than 5, more desirably equal to or greater than 10; and/or
wherein the particulate comprises Magnesium stearate, Magnesium palmitate or mixtures thereof, in particular crystalline Magnesium stearate, Magnesium palmitate or mixtures thereof, more particularly crystalline Magnesium stearate, Magnesium palmitate or mixtures having a d-spacing equal to or greater than 10.

38. A device according to claim 26, wherein the medicinal inhalation device is a pressurized metered dose medicinal inhalation device, in particular a pressurized metered dose inhaler.
